(19) United States
(12) Patent Application Publication
(10) Pub. No.: US 2024/0173206 A1
(43) Pub. Date: May 30, 2024

(54) WEARABLE TRANSCUTANEOUS ELECTRICAL ACUPOINT STIMULATION PHYSIOTHERAPY DEVICE FOR PREVENTING AND TREATING POSTOPERATIVE COGNITIVE DYSFUNCTION

(71) Applicant: Air Force Medical University, Xi'an (CN)

(72) Inventors: Changjun GAO, Xi'an (CN); Fei GUO, Xi'an (CN); Ruili HAN, Xi'an (CN); Li SUN, Xi'an (CN); Yuan QIN, Xi'an (CN)

(21) Appl. No.: 18/502,451

(22) Filed: Nov. 6, 2023

(30) Foreign Application Priority Data
Nov. 28, 2022 (CN) 202211499629.6

(51) Int. Cl. A61H 39/00

(52) U.S. Cl. CPC A61H 39/002; A61H 2201/165; A61H 2205/025; A61H 2205/102

(57) ABSTRACT

Provided is a wearable transcutaneous electrical acupoint stimulation physiotherapy device for preventing and treating postoperative cognitive dysfunction, including a head-mounted wearable device, a hand wearable device and a knee wearable device; where the head-mounted wearable device includes a forehead-shaped piece, a head coronal plane adjustable ring elastic belt, a head sagittal plane adjustable ring elastic belt, a first slidable electrode fixing block, a first electrode pad and a first sliding guide groove.

### CROSS-REFERENCE TO RELATED APPLICATIONS

This application claims priority of Chinese Patent Application No. 202211499629.6, filed on Nov. 28, 2022, the entire contents of which are incorporated herein by reference.

### TECHNICAL FIELD

The disclosure relates to the technical field of therapeutic devices for patients without self-consciousness, and in particular to a wearable transcutaneous electrical acupoint stimulation physiotherapy device for preventing and treating postoperative cognitive dysfunction.

### BACKGROUND

At present, there are two main ways for electro-acupuncture therapeutic apparatus to act on patients: one is to connect filiform needle with the electrode circuit of electro-acupuncture therapeutic apparatus after needling acupoints and obtaining needling reaction (Deqi), and the other is to connect electrode pads with the electrode circuit of electro-acupuncture therapeutic apparatus after sticking the electrode pads on target acupoints and then adjust relevant parameters such as waveform, frequency, current intensity and action duration for realizing the therapeutic effects.

With the continuous improvement of electro-acupuncture therapy technology, its research and application in clinical patients have been greatly developed, and more and more evidence-based medical data have confirmed the reliability of electro-acupuncture technology in preventing and treating postoperative cognitive dysfunction in elderly patients.

However, only the professional medical staff of acupuncture and moxibustion in traditional Chinese medicine may accurately pierce filiform needle into the acupoints, which requires high standard operation and may cause bad experiences such as skin damage, infection and pain stimulation. Therefore, it is not easy for ordinary patients to accept and may not be widely popularized and applied.

In the transcutaneous electrical acupoint stimulation technology, the electrode pads are attached to the patient's acupoints and then connected to the electro-acupuncture therapeutic apparatus. This technology is welcomed by clinical patients because of its simpler non-invasive operation, but only professionals with medical background may accurately locate the acupoints, the electrode pads need to be repeatedly attached to different target acupoints every time, the electrode circuit is long and the connection process is very complicated, and some target acupoints often fall off because of more hair and epidermal secretions.

At present, when the electro-acupuncture therapeutic apparatus is applied to perioperative brain protection, the selected target compatible acupoints are Baihui, Fengfu, Hegu, Neiguan and Zusanli points, and the selected action time is mostly 30 minutes a day from 3 days before operation to 1 week after operation. Relevant researches show that this treatment may significantly reduce the incidence of postoperative cognitive dysfunction, decrease hospitalization time and postoperative complications, further reduce the economic burden of patients and generate good economic and social benefits.

However, in the actual operation process, the researchers find that when the electrode pads are stuck to Baihui and Fengfu points, the electrode pads may not be closely attached to the target acupoints because of the hair on the stuck acupoints, even with the help of elastic headgear and other equipment, as the patient's posture changes consciously or unconsciously during perioperative period, the electrode pads are very easy to get out of contact with the acupoints and out of function, lines are easy to be knotted and torn off, and medical staff often feel electric shock when they inadvertently touch the electrode pads exposed on the patient's body surface; moreover, as the electrode pads need to be pasted repeatedly every time, the workload of medical staff increases and the electrode pads are wasted.

Therefore, it is urgent to develop a wearable transcutaneous electrical acupoint stimulation physiotherapy device with reusability, strong stability, good adhesion to the target acupoints and embedded circuit, so as to popularize the transcutaneous electrical acupoint stimulation technology to prevent and treat postoperative cognitive dysfunction of patients.

### SUMMARY

An objective of the present disclosure is to provide a wearable transcutaneous electrical acupoint stimulation physiotherapy device for preventing and treating postoperative cognitive dysfunction, so as to solve the existing problems, that is, when the electrode pads are stuck to Baihui and Fengfu points, the electrode pads may not be closely attached to the target acupoints because of the hair on the stuck acupoints, even with the help of elastic headgear and other equipment, and as the patient changes posture consciously or unconsciously during perioperative period, the electrode pads are very easy to get out of contact with the acupoints and out of function, lines are easy to be knotted and torn off, and medical staff often feel electric shock when they inadvertently touch the electrode pads exposed on the patient's body surface; moreover, as the electrode pads need to be pasted repeatedly every time, the workload of medical staff increases and the electrode pads are wasted.

To achieve the above objective, the present disclosure adopts the following schema.

Optionally, a wearable transcutaneous electrical acupoint stimulation physiotherapy device for preventing and treating postoperative cognitive dysfunction includes a head-mounted wearable device, a hand wearable device and a knee wearable device; where the head-mounted wearable device includes a forehead-shaped piece, a head coronal plane adjustable ring elastic belt, a head sagittal plane adjustable ring elastic belt, a first slidable electrode fixing block, a first electrode pad and a first sliding guide groove; both ends of the forehead-shaped piece are connected with the head coronal plane adjustable ring elastic belt, one end of the head coronal plane adjustable ring elastic belt away from the forehead-shaped piece is fixedly connected with a back pillow gasket, a top end of the back pillow gasket and a top end of the forehead-shaped piece are both fixedly connected with the head sagittal plane adjustable ring elastic belt, a top end of the head sagittal plane adjustable ring elastic belt is provided with the first sliding guide groove, an outside of the first sliding guide groove is slidably connected with the first slidable electrode fixing block, and a top surface of the first slidable electrode fixing block is embedded with the first electrode pad.

Optionally, the hand wearable device includes a wrist module, a palm module and a linkage elastic belt, where one end of the linkage elastic belt is connected with the wrist module, and an other end of the linkage elastic belt is connected with the palm module.

Optionally, the palm module includes a hand back adjustable guided shaped piece, a palm adjustable ring elastic belt, a second slidable electrode fixing block, a second electrode pad and a second sliding guide groove, where top ends of the hand back adjustable guided shaped piece is connected with the palm adjustable ring elastic belt, an inner surface of the hand back adjustable guided shaped piece is provided with the second sliding guide groove, an outside of the hand back adjustable guided shaped piece is slidably connected with the second slidable electrode fixing block, and a top surface of the second slidable electrode fixing block is fixedly connected with the second electrode pad.

Optionally, the wrist module includes a wrist adjustable guided shaped piece, a third slidable electrode fixing block, a third electrode pad and a wrist adjustable ring elastic belt, where bottom ends of the wrist adjustable guided shaped piece are connected with the wrist adjustable ring elastic belt, an outside of the wrist adjustable guided shaped piece is slidably connected with the third slidable electrode fixing block, and a top surface of the third slidable electrode fixing block is connected with the third electrode pad.

Optionally, the knee wearable device includes a knee coronal plane adjustable ring elastic belt, a knee coronal plane adjustable guided shaped piece, a third sliding guide groove, a fourth slidable electrode fixing block and a fourth electrode pad, where both ends of the knee coronal plane adjustable guided shaped piece are connected with the knee coronal plane adjustable ring elastic belt, an outer surface of the knee coronal plane adjustable guided shaped piece is provided with the third sliding guide groove, and an outside of the knee coronal plane adjustable guided shaped piece is slidably connected with the fourth slidable electrode fixing block, and an outer surface of the fourth slidable electrode fixing block is fixedly connected with the fourth electrode pad.

Optionally, the forehead-shaped piece, the head sagittal plane adjustable ring elastic belt, the hand back adjustable guided shaped piece, the wrist adjustable guided shaped piece and the knee coronal plane adjustable guided shaped piece are all made of engineering plastics.

Optionally, an inside of the first sliding guide groove is provided with a first adjusting groove, an inside of the first adjusting groove is embedded with a first embedded electrode copper wire, the first embedded electrode copper wire is in clearance fit with the first adjusting groove, one end of the first embedded electrode copper wire is connected with the first electrode pad, and one end of the first embedded electrode copper wire away from the first electrode pad is connected with a transcutaneous electrical acupoint stimulation therapeutic instrument.

Optionally, second embedded electrode copper wires are embedded in an inside of the second sliding guide groove and an inside of the wrist adjustable guided shaped piece respectively, second adjusting grooves are provided in the inside of the second sliding guide groove and the inside of the wrist adjustable guided shaped piece respectively, an inside of each of the second adjusting grooves is embedded with corresponding one of the second embedded electrode copper wires, each of the second embedded electrode copper wires is in clearance fit with corresponding one of the second adjusting grooves, ends of the second embedded electrode copper wires are connected with the third electrode pad and the second electrode pad respectively, and other ends of the second embedded electrode copper wires away from the second electrode pad and the third electrode pad are electrically connected with a transcutaneous electrical acupoint stimulation therapeutic instrument.

Optionally, an inside of the third sliding guide groove is provided with a third adjusting groove, an inside of the third adjusting groove is embedded with a third embedded electrode copper wire, the third embedded electrode copper wire is in clearance fit with the third adjusting groove, one end of the third embedded electrode copper wire is connected with the fourth electrode pad, and one end of the third embedded electrode copper wire away from the fourth electrode pad is connected with a transcutaneous electrical acupoint stimulation therapeutic instrument.

The embodiments have the following effects.

The disclosure may be applied to patients with or without self-consciousness, and may make the electrodes closely adhere to the patient's Baihui, Fengfu and bilateral Hegu, Neiguan and Zusanli points. The disclosure has the advantages of simple operation, accurate positioning, reusability, good patient compliance, safety and comfort, and may be used by people with or without medical knowledge background according to the instructions. Therefore, the disclosure has great popularization and application value and may effectively improve the perioperative brain health level of patients.

### BRIEF DESCRIPTION OF THE DRAWINGS

In order to more clearly explain the embodiments of the present disclosure, the following will briefly introduce the drawings to be used in the embodiments. Obviously, the drawings in the following description are only some embodiments of the present disclosure. For those of ordinary skill in the art, other drawings may be obtained according to these drawings without any creative effort.

FIG. 1 is a schematic structural diagram of a head-mounted wearable device of the present disclosure.

FIG. 2 is a schematic structural diagram of a hand wearable device of the present disclosure.

FIG. 3 is a schematic structural diagram of a knee wearable device of the present disclosure.

### DETAILED DESCRIPTION OF THE EMBODIMENTS

In the following, the technical scheme in the embodiment of the disclosure is clearly and completely described with reference to the drawings in the embodiment of the disclosure. Obviously, the described embodiment is only a part of the embodiments of the disclosure, but not the whole embodiments.

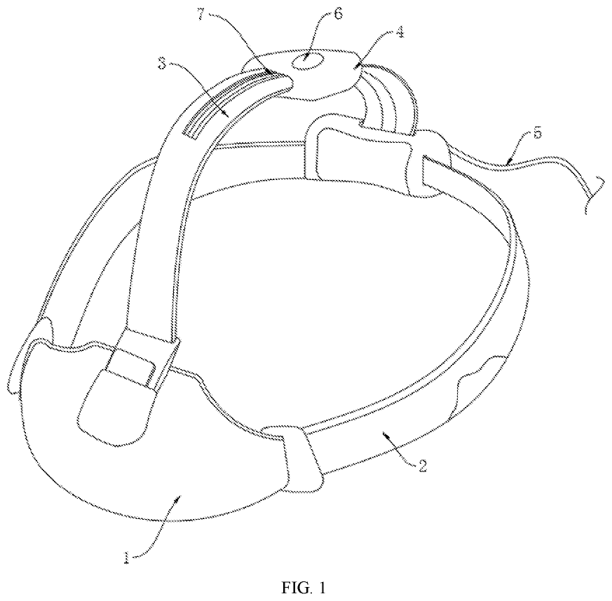

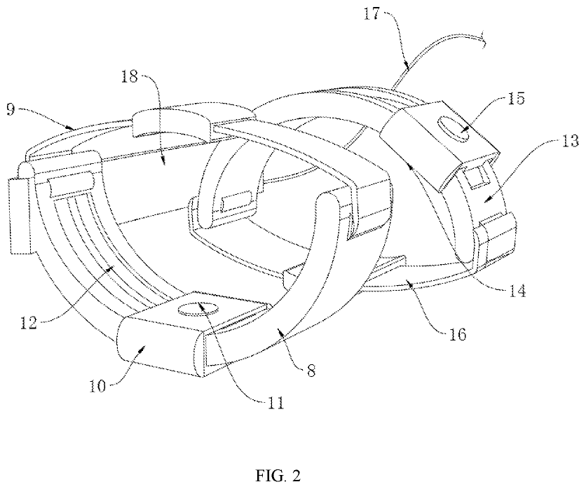

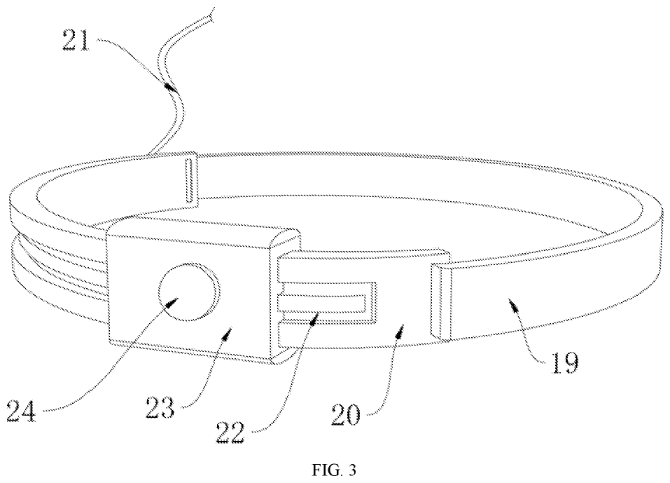

Referring to FIG. 1, FIG. 2 and FIG. 3, a wearable transcutaneous electrical acupoint stimulation physiotherapy device for preventing and treating postoperative cognitive dysfunction includes a head-mounted wearable device, a hand wearable device and a knee wearable device; where the head-mounted wearable device includes a forehead-shaped piece 1, a head coronal plane adjustable ring elastic belt 2, a head sagittal plane adjustable ring elastic belt 3, a first slidable electrode fixing block 4, a first electrode pad 6 and a first sliding guide groove 7; both ends of the forehead-shaped piece 1 are connected with the head coronal plane adjustable ring elastic belt 2, one end of the head coronal plane adjustable ring elastic belt 2 away from the forehead-shaped piece 1 is fixedly connected with a back pillow gasket, a top end of the back pillow gasket and a top end of the forehead-shaped piece 1 are both fixedly connected with the head sagittal plane adjustable ring elastic belt 3, a top end of the head sagittal plane adjustable ring elastic belt 3 is provided with the first sliding guide groove 7, an outside of the first sliding guide groove 7 is slidably connected with the first slidable electrode fixing block 4, and a top surface of the first slidable electrode fixing block 4 is embedded with the first electrode pad 6.

The hand wearable device includes a wrist module, a palm module and a linkage elastic belt 18, where one end of the linkage elastic belt 18 is connected with the wrist module, and an other end of the linkage elastic belt 18 is connected with the palm module.

The palm module includes a hand back adjustable guided shaped piece 8, a palm adjustable ring elastic belt 9, a second slidable electrode fixing block 10, a second electrode pad 11 and a second sliding guide groove 12, where top ends of the hand back adjustable guided shaped piece 8 is connected with the palm adjustable ring elastic belt 9, an inner surface of the hand back adjustable guided shaped piece 8 is provided with the second sliding guide groove 12, an outside of the hand back adjustable guided shaped piece 8 is slidably connected with the second slidable electrode fixing block 10, and a top surface of the second slidable electrode fixing block 10 is fixedly connected with the second electrode pad 11.

The wrist module includes a wrist adjustable guided shaped piece 13, a third slidable electrode fixing block 14, a third electrode pad 15 and a wrist adjustable ring elastic belt 16, where bottom ends of the wrist adjustable guided shaped piece 13 are connected with the wrist adjustable ring elastic belt 16, an outside of the wrist adjustable guided shaped piece 13 is slidably connected with the third slidable electrode fixing block 14, and a top surface of the third slidable electrode fixing block 14 is connected with the third electrode pad 15.

The knee wearable device includes a knee coronal plane adjustable ring elastic belt 19, a knee coronal plane adjustable guided shaped piece 20, a third sliding guide groove 22, a fourth slidable electrode fixing block 23 and a fourth electrode pad 24, where both ends of the knee coronal plane adjustable guided shaped piece 20 are connected with the knee coronal plane adjustable ring elastic belt 19, an outer surface of the knee coronal plane adjustable guided shaped piece 20 is provided with the third sliding guide groove 22, and an outside of the knee coronal plane adjustable guided shaped piece 20 is slidably connected with the fourth slidable electrode fixing block 23, and an outer surface of the fourth slidable electrode fixing block 23 is fixedly connected with the fourth electrode pad 24.

The forehead-shaped piece 1, the head sagittal plane adjustable ring elastic belt 3, the hand back adjustable guided shaped piece 8, the wrist adjustable guided shaped piece 13 and the knee coronal plane adjustable guided shaped piece 20 are all made of engineering plastics.

An inside of the first sliding guide groove 7 is provided with a first adjusting groove, an inside of the first adjusting groove is embedded with a first embedded electrode copper wire 5, the first embedded electrode copper wire 5 is in clearance fit with the first adjusting groove, one end of the first embedded electrode copper wire 5 is connected with the first electrode pad 6, and one end of the first embedded electrode copper wire 5 away from the first electrode pad 6 is connected with a transcutaneous electrical acupoint stimulation therapeutic instrument.

Second embedded electrode copper wires 17 are embedded in an inside of the second sliding guide groove 12 and an inside of the wrist adjustable guided shaped piece 13 respectively, second adjusting grooves are provided in the inside of the second sliding guide groove 12 and the inside of the wrist adjustable guided shaped piece 13 respectively, an inside of each of the second adjusting grooves is embedded with corresponding one of the second embedded electrode copper wires 17, each of the second embedded electrode copper wires 17 is in clearance fit with corresponding one of the second adjusting grooves, ends of the second embedded electrode copper wires 17 are connected with the third electrode pad 15 and the second electrode pad 11 respectively, and other ends of the second embedded electrode copper wires 17 away from the second electrode pad 11 and the third electrode pad 15 are electrically connected with a transcutaneous electrical acupoint stimulation therapeutic instrument.

An inside of the third sliding guide groove 22 is provided with a third adjusting groove, an inside of the third adjusting groove is embedded with a third embedded electrode copper wire 21, the third embedded electrode copper wire 21 is in clearance fit with the third adjusting groove, one end of the third embedded electrode copper wire 21 is connected with the fourth electrode pad 24, and one end of the third embedded electrode copper wire 21 away from the fourth electrode pad 24 is connected with a transcutaneous electrical acupoint stimulation therapeutic instrument.

Through the cooperation of the head coronal plane adjustable ring elastic belt 2 and the forehead-shaped piece 1, the head coronal plane adjustable ring elastic belt 2 is adjusted to adapt to the size of the patient's head circumference, so that the head-mounted wearable device is tightly fixed on the patient's head; the first slidable electrode fixing block 4 is tightly contacted with the patient's scalp through the compression of the head sagittal plane adjustable ring elastic belt 3; through the cooperation of the first sliding guide groove 7 and the head sagittal plane adjustable ring elastic belt 3, a position of the first electrode pad 6 is adjusted in a sliding way, so that the first electrode pad 6 is accurately positioned above Baihui acupoint, the first embedded electrode copper wire 5 is connected to the transcutaneous electrical acupoint stimulation therapeutic instrument, the transcutaneous electrical acupoint stimulation therapeutic instrument is electrified, the related parameters are adjusted, and the treatment is started.

Through the cooperation of the second sliding guide groove 12 and the palm adjustable ring elastic belt 9, the ring formed by the second sliding guide groove 12 and the palm adjustable ring elastic belt 9 is tightly fixed on the palm of the patient, so that the second electrode pad 11 is in close contact with the skin of the tiger's mouth of the patient, and then the second slidable electrode fixing block 10 on the second sliding guide groove 12 is slid to ensure that the second electrode pad 11 is located above the Hegu point. Through the cooperation between the wrist adjustable ring elastic belt 16 and the wrist adjustable guided shaped piece 13, the ring formed by the wrist adjustable ring elastic belt 16 and the wrist adjustable guided shaped piece 13 is fixed on the wrist of the patient; the wrist adjustable ring elastic belt 16 has elasticity, and may be adjusted to adapt to different wrist sizes, and the third slidable electrode fixing block 14 on the wrist adjustable guided shaped piece 13 is slidably adjusted, so that the third electrode pad 15 is located above the Neiguan point and is in close contact with the skin of the wrist of the patient; the second embedded electrode copper wire 17 is connected to the transcutaneous electrical acupoint stimulation therapeutic instrument, the transcutaneous electrical acupoint stimulation therapeutic instrument is electrified, the related parameters are adjusted, and the treatment is started.

Through the cooperation of the knee coronal plane adjustable guided shaped piece 20 and the knee coronal plane adjustable ring elastic belt 19, the knee wearable device is tightly fixed under the knee joint of the patient by adjusting the knee coronal plane adjustable ring elastic belt 19, so that the fourth electrode pad 24 is in close contact with the knee skin. The fourth slidable electrode fixing block 23 is slidably adjusted above Zusanli point, so that the fourth electrode pad 24 is in close contact with the Zusanli point. At this time, the third embedded electrode copper wire 21 is connected to the transcutaneous electrical acupoint stimulation therapeutic instrument, the transcutaneous electrical acupoint stimulation therapeutic instrument is electrified, the related parameters are adjusted, and the treatment is started.

It should be understood that the technical schemes of the present disclosure are not limited to the above specific embodiments, and any technical variations made according to the technical solutions of the present disclosure, without departing from the protection scope defined by claims of the present disclosure, shall fall within the scope of protection of the present disclosure.

What is claimed is:

1. A wearable transcutaneous electrical acupoint stimulation physiotherapy device for preventing and treating postoperative cognitive dysfunctions, comprising a head-mounted wearable device, a hand wearable device and a knee wearable device; wherein the head-mounted wearable device comprises a forehead-shaped piece, a head coronal plane adjustable ring elastic belt, a head sagittal plane adjustable ring elastic belt, a first slidable electrode fixing block, a first electrode pad and a first sliding guide groove; both ends of the forehead-shaped piece are connected with the head coronal plane adjustable ring elastic belt, one end of the head coronal plane adjustable ring elastic belt away from the forehead-shaped piece is fixedly connected with a back pillow gasket, a top end of the back pillow gasket and a top end of the forehead-shaped piece are both fixedly connected with the head sagittal plane adjustable ring elastic belt, a top end of the head sagittal plane adjustable ring elastic belt is provided with the first sliding guide groove, an outside of the first sliding guide groove is slidably connected with the first slidable electrode fixing block, and a top surface of the first slidable electrode fixing block is embedded with the first electrode pad.

2. The wearable transcutaneous electrical acupoint stimulation physiotherapy device for preventing and treating postoperative cognitive dysfunctions according to claim 1, wherein the hand wearable device comprises a wrist module, a palm module and a linkage elastic belt, one end of the linkage elastic belt is connected with the wrist module, and an other end of the linkage elastic belt is connected with the palm module.

3. The wearable transcutaneous electrical acupoint stimulation physiotherapy device for preventing and treating postoperative cognitive dysfunctions according to claim 2, wherein the palm module comprises a hand back adjustable guided shaped piece, a palm adjustable ring elastic belt, a second slidable electrode fixing block, a second electrode pad and a second sliding guide groove, wherein top ends of the hand back adjustable guided shaped piece is connected with the palm adjustable ring elastic belt, an inner surface of the hand back adjustable guided shaped piece is provided with the second sliding guide groove, an outside of the hand back adjustable guided shaped piece is slidably connected with the second slidable electrode fixing block, and a top surface of the second slidable electrode fixing block is fixedly connected with the second electrode pad.

4. The wearable transcutaneous electrical acupoint stimulation physiotherapy device for preventing and treating postoperative cognitive dysfunctions according to claim 3, wherein the wrist module comprises a wrist adjustable guided shaped piece, a third slidable electrode fixing block, a third electrode pad and a wrist adjustable ring elastic belt, wherein bottom ends of the wrist adjustable guided shaped piece are connected with the wrist adjustable ring elastic belt, an outside of the wrist adjustable guided shaped piece is slidably connected with the third slidable electrode fixing block, and a top surface of the third slidable electrode fixing block is connected with the third electrode pad.

5. The wearable transcutaneous electrical acupoint stimulation physiotherapy device for preventing and treating postoperative cognitive dysfunctions according to claim 4, wherein the knee wearable device comprises a knee coronal plane adjustable ring elastic belt, a knee coronal plane adjustable guided shaped piece, a third sliding guide groove, a fourth slidable electrode fixing block and a fourth electrode pad, wherein both ends of the knee coronal plane adjustable guided shaped piece are connected with the knee coronal plane adjustable ring elastic belt, an outer surface of the knee coronal plane adjustable guided shaped piece is provided with the third sliding guide groove, and an outside of the knee coronal plane adjustable guided shaped piece is slidably connected with the fourth slidable electrode fixing block, and an outer surface of the fourth slidable electrode fixing block is fixedly connected with the fourth electrode pad.

6. The wearable transcutaneous electrical acupoint stimulation physiotherapy device for preventing and treating postoperative cognitive dysfunctions according to claim 5, wherein the forehead-shaped piece, the head sagittal plane adjustable ring elastic belt, the hand back adjustable guided shaped piece, the wrist adjustable guided shaped piece and the knee coronal plane adjustable guided shaped piece are all made of engineering plastics.

7. The wearable transcutaneous electrical acupoint stimulation physiotherapy device for preventing and treating postoperative cognitive dysfunctions according to claim 1, wherein an inside of the first sliding guide groove is provided with a first adjusting groove, an inside of the first adjusting groove is embedded with a first embedded electrode copper wire, the first embedded electrode copper wire is in clearance fit with the first adjusting groove, one end of the first embedded electrode copper wire is connected with the first electrode pad, and one end of the first embedded electrode copper wire away from the first electrode pad is connected with a transcutaneous electrical acupoint stimulation therapeutic instrument.

8. The wearable transcutaneous electrical acupoint stimulation physiotherapy device for preventing and treating postoperative cognitive dysfunctions according to claim 4, wherein second embedded electrode copper wires are embedded in an inside of the second sliding guide groove and an inside of the wrist adjustable guided shaped piece respectively, second adjusting grooves are provided in the inside of the second sliding guide groove and the inside of the wrist adjustable guided shaped piece respectively, an inside of each of the second adjusting grooves is embedded with corresponding one of the second embedded electrode copper wires, each of the second embedded electrode copper wires is in clearance fit with corresponding one of the second adjusting grooves, ends of the second embedded electrode copper wires are connected with the third electrode pad and the second electrode pad respectively, and other ends of the second embedded electrode copper wires away from the second electrode pad and the third electrode pad are electrically connected with a transcutaneous electrical acupoint stimulation therapeutic instrument.

9. The wearable transcutaneous electrical acupoint stimulation physiotherapy device for preventing and treating postoperative cognitive dysfunctions according to claim 5, wherein an inside of the third sliding guide groove is provided with a third adjusting groove, an inside of the third adjusting groove is embedded with a third embedded electrode copper wire, the third embedded electrode copper wire is in clearance fit with the third adjusting groove, one end of the third embedded electrode copper wire is connected with the fourth electrode pad, and one end of the third embedded electrode copper wire away from the fourth electrode pad is connected with a transcutaneous electrical acupoint stimulation therapeutic instrument.
